(19) United States
(12) Patent Application Publication
(10) Pub. No.: US 2008/0172050 A1
(43) Pub. Date: Jul. 17, 2008

(54) RADIOFREQUENCY THERMAL BALLOON CATHETER SYSTEM

(75) Inventor: Shutaro Satake, Kanagawa (JP)

Correspondence Address: DARBY & DARBY P.C., P.O. BOX 770, Church Street Station, New York, NY, 10008-0770, US

(73) Assignee: JAPAN ELECTEL INC., Tokyo (JP)

(21) Appl. No.: 11/838,426

(22) Filed: Aug. 14, 2007

(30) Foreign Application Priority Data
Jan. 12, 2007 (JP) 2007-004130

(51) Int. Cl. A61B 18/18

(52) U.S. Cl. 606/33

(57) ABSTRACT

A radiofrequency thermal balloon catheter system which is accurate in the detection temperature, capable of miniaturization compared to conventional systems, reliably detecting pinholes and adhesion of thrombi. A thermocouple is composed of a radiofrequency transmission line and a single metal wire joined to the distal end thereof. A coiled electrode is formed in a coil shape by extending the distal end of the radiofrequency transmission line. The distal end of the metal wire is brought into pin-point connection with the proximal end of the coiled electrode. A radiofrequency generator monitors the radiofrequency output, total impedance, and reflection waves, while supplying the coiled electrode and a counter electrode plate with a radiofrequency of 1 to 5 MHz, and automatically controls the radiofrequency output, so that the temperature of the coiled electrode can be kept at a target value.

### INCORPORATION BY REFERENCE

The present application claims priority under 35 U.S.C. §119 to Japanese Patent Application No. 2007-004130 filed on Jan. 12, 2007. The content of the application is incorporated herein by reference in its entirety.

### BACKGROUND OF THE INVENTION

1. Field of the Invention

The present invention relates to a radiofrequency thermal balloon catheter system used for treatment of cardiovascular diseases.

2. Description of Related Art

A method is proposed by the present inventor in which, with respect to lesions such as an origin of arrhythmia or atherosclerosis, an electrode for delivery of radiofrequency energy is arranged inside an elastic balloon, and a radiofrequency electric field is radiated therefrom, to provide thermotherapy to a tissue in contact with the balloon, as is shown in: Japanese Unexamined Patent Publication No. 2005-177293; Japanese Unexamined Patent Publication No. 2004-223080; Japanese Patent Publication No. 2538375; Japanese Patent Publication No. 2510428; Japanese Patent Publication No. 2574119; U.S. Pat. No. 6,491,710 B2 issued on Dec. 10, 2002; U.S. Pat. No. 6,952,615 B2, issued on Oct. 4, 2005; and U.S. Pat. No. 7,112,198 B2, issued on Sep. 26, 2006.

Generally, a thermocouple is used as a device for measuring temperature which detects the temperature inside a balloon, and this thermocouple is provided by bonding with an electrode for delivery of radiofrequency energy. In this case, if for some reason the thermocouple is detached from the electrode for delivery of radiofrequency energy, the temperature will be detected at a site different from the site to be measured, causing a problem in that the detection temperature becomes inaccurate.

Moreover, if a small tissue is to be a target, the catheter needs to be miniaturized. However, the miniaturization is limited since the inside of the balloon catheter needs to be arranged with, in addition to the electrode for delivery of radiofrequency energy, a thermocouple for monitoring the temperature inside the balloon, with two lead lines such as two coated wires of a copper wire and a constantan wire.

Furthermore, a conventional radiofrequency thermal balloon catheter system has been unable to detect phenomena of pinholes in the balloon and adhesion of thrombi on the balloon surface. If a pinhole is made in the balloon, radiofrequency currents are concentrated in the part where the pinhole is made, causing concern of excessive cauterization. Moreover, if thrombi are adhered on the balloon surface, there is concern of causing thromboembolism. Thus, a system which can reliably detect these phenomena has been in demand.

### SUMMARY OF THE INVENTION

Therefore, the present invention takes the above problems into consideration, with an object of providing a radiofrequency thermal balloon catheter system which is accurate in the detection temperature, is capable of miniaturization compared to conventional systems, and is capable of reliably detecting pinholes and adhesion of thrombi.

The radiofrequency thermal balloon catheter system of the present invention includes: a catheter shaft composed of an outer tube and an inner tube which are slidable with each other; a balloon provided between a distal end of the outer tube and the vicinity of a distal end of the inner tube; a unipolar electrode inside of this balloon electrode for delivery of radiofrequency energy; a radiofrequency transmission line connected to this unipolar electrode; a thermocouple which detects a temperature of the unipolar electrode; a solution sending duct formed between the outer tube and the inner tube, in communication with an inside of the balloon; a vibration generator which applies vibrational waves to the balloon via this solution sending duct; a thermometer which indicates a temperature detected by the thermocouple; a high-frequency cut filter which is provided between this thermometer and the thermocouple, and cuts a high-frequency component input into the thermometer; a radiofrequency generator which supplies the radiofrequency transmission line and a counter electrode plate provided outside of the balloon, with a radiofrequency; and a low-frequency cut filter which is provided between this radiofrequency generator and the radiofrequency transmission line, and cuts a low-frequency component of the radiofrequency output from the radiofrequency generator; and

the thermocouple composed of the radiofrequency transmission line and a single superfine dissimilar metal wire joined to a distal end of the radiofrequency transmission line, and

the radiofrequency generator: is designed to be capable of monitoring a radiofrequency output, a total impedance that is the sum total of an internal balloon impedance, a balloon membrane impedance, and a tissue impedance, and reflection waves, while supplying the unipolar electrode and the counter electrode plate with a radiofrequency of 1 to 5 MHz: and further is designed to automatically control the radiofrequency output, so that the temperature of the unipolar electrode can be kept at a target value.

Moreover, the radiofrequency thermal balloon catheter system of the present invention is characterized in that the unipolar electrode is a coiled electrode formed in a coil shape by extending the distal end of the radiofrequency transmission line, and the distal end of the dissimilar metal wire is brought into pin-point connection with the proximal end of the coiled electrode.

Furthermore, the radiofrequency thermal balloon catheter system of the present invention is characterized in that the radiofrequency generator is designed to indicate an alarm showing a pinhole occurrence in the membrane of the balloon, or to automatically stop supplying a radiofrequency, when the total impedance is decreased by more than a fixed value with reference to a steady-state value.

Moreover, the radiofrequency thermal balloon catheter system of the present invention is characterized in that the radiofrequency generator is designed to indicate an alarm showing thrombus formation on the membrane of the balloon, or to automatically stop supplying a radiofrequency, when the total impedance is increased by more than a fixed value with reference to a steady-state value.

According to the radiofrequency thermal balloon catheter system of the present invention, since the thermocouple comprises the radiofrequency transmission line and the single dissimilar metal wire joined to the distal end of the radiofrequency transmission line, then if the dissimilar metal wire is disconnected from the radiofrequency transmission line, the measurement becomes impossible and the breakdown can be immediately judged. Consequently, the conventional problem in that, if for some reason the thermocouple is detached from the unipolar electrode for delivery of radiofrequency energy, the temperature will be detected in a location different from the location to be measured, and thereby the detection temperature becomes inaccurate, can be solved.

Moreover, by the above structure, one of the dissimilar metal wires constituting the thermocouple is used jointly as the radiofrequency transmission line. Consequently, compared to a conventional case where a thermocouple is independently arranged, one wire can be omitted, and thus the catheter can be miniaturized by omitting the space for arranging the thermocouple.

Furthermore, according to the radiofrequency thermal balloon catheter system of the present invention, since the unipolar electrode is a coiled electrode formed in a coil shape by extending the distal end of the radiofrequency transmission line, and the distal end of the dissimilar metal wire is brought into pin-point connection with the proximal end of the coiled electrode, then the formation thereof is easy, and the connection part of the distal end of the dissimilar metal wire is reliably located inside of the balloon, and thus the detection temperature becomes accurate. Moreover, since the thermocouple formed by bringing the distal end of the superfine dissimilar metal wire into pin-point connection with the proximal end of the unipolar electrode, has a small heat capacity, the temperature of the basal part of the unipolar electrode can be accurately and instantaneously detected.

Moreover, according to the radiofrequency thermal balloon catheter system of the present invention, the radiofrequency generator is designed to be capable of monitoring the radiofrequency output, the total impedance, and reflection waves, while supplying the unipolar electrode and the counter electrode plate with a radiofrequency of 1 to 5 MHz, and further is designed to automatically control the radiofrequency output, so that the temperature of the unipolar electrode can be kept at a target value. Therefore, even if the catheter is miniaturized, the inside of the balloon can be efficiently heated and the handling can be facilitated.

Furthermore, according to the radiofrequency thermal balloon catheter system of the present invention, the radiofrequency generator is designed to indicate an alarm showing a pinhole occurrence in the membrane of the balloon, or to automatically stop supplying a radiofrequency, when the total impedance is decreased by a fixed value with reference to a steady-state value. Therefore, the phenomenon where a pinhole occurs with the impedance drop, can be reliably detected, and excessive cauterization can be prevented.

Moreover, according to the radiofrequency thermal balloon catheter system of the present invention, the radiofrequency generator is designed to indicate an alarm showing thrombus formation on the membrane of the balloon, or to automatically stop supplying a radiofrequency, when the total impedance is increased by more than a fixed value with reference to a steady-state value. Therefore, the phenomenon, where a thrombus is adhered with the impedance rise, can be reliably detected, and thromboembolism can be prevented.

### BRIEF DESCRIPTION OF THE DRAWINGS

FIG. 1 is an enlarged fragmentary view of the vicinity of a balloon showing a first embodiment of a radiofrequency thermal balloon catheter system of the present invention.

FIG. 2 is an overall diagram showing the balloon placed in a laboratory bath.

FIG. 3 is a graph showing changes of impedance and the like, accompanying pinhole occurrence in the balloon.

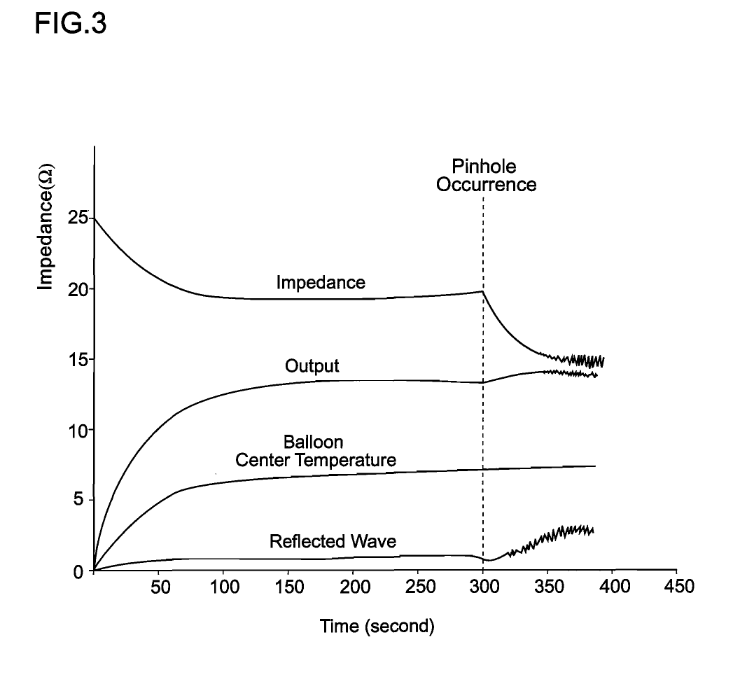

FIG. 4 is a graph showing changes of impedance and the like, accompanying thrombus formation in the balloon.

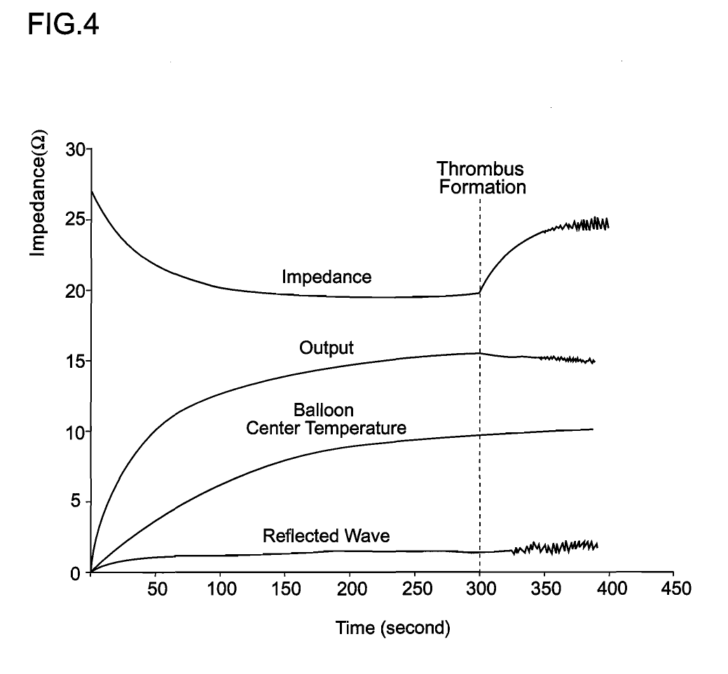

### DETAILED DESCRIPTION OF THE INVENTION

Hereunder is a detailed description of an embodiment of the radiofrequency thermal balloon catheter system of the present invention, with reference to the appended drawings.

### Embodiment 1

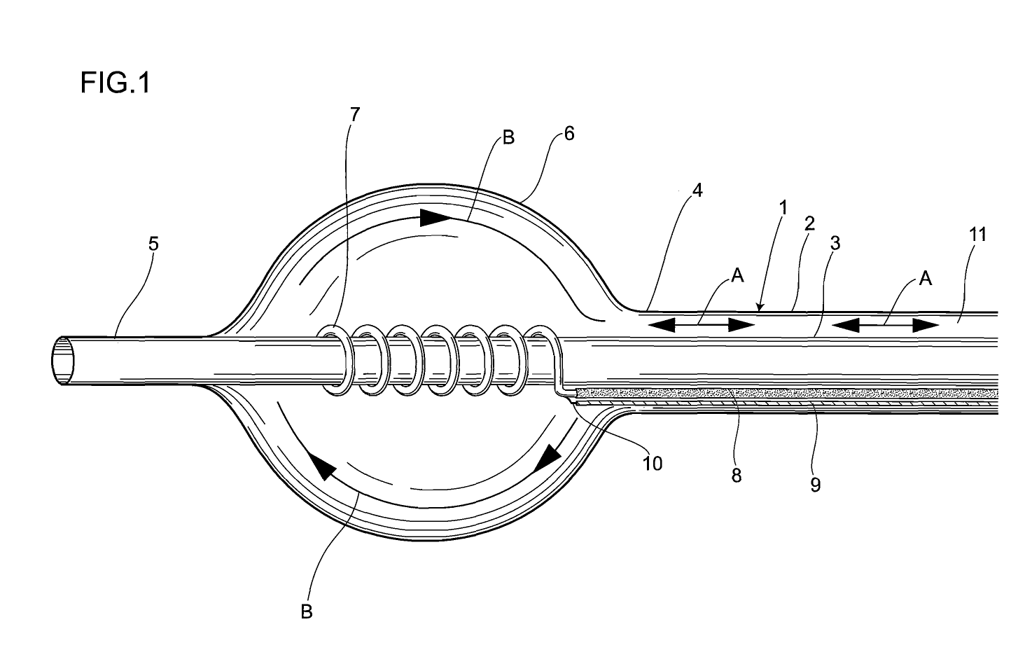

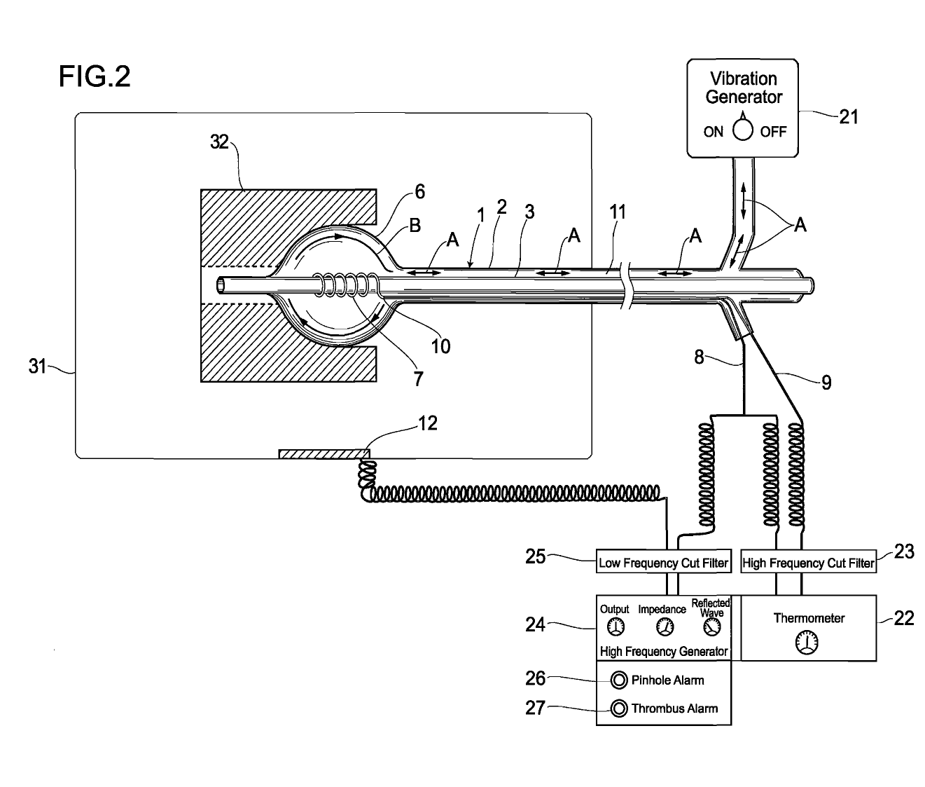

The structure of the radiofrequency thermal balloon catheter system of the present embodiment is described, with reference to FIG. 1 and FIG. 2.

Reference symbol 1 denotes a catheter shaft. This catheter shaft 1 is composed of an outer tube 2 and an inner tube 3 which are slidable with each other. A balloon 6 is provided between a distal end 4 of the outer tube 2 and the vicinity of the distal end 5 of the inner tubes.

A coiled electrode 7 serving as a unipolar electrode for delivery of radiofrequency energy is provided inside of the balloon 6. Moreover, a radiofrequency transmission line 8 is connected to the coiled electrode 7. More specifically, the coiled electrode 7 is formed in a coil shape extending from the distal end of the radiofrequency transmission line 8, and is wound around the inner tube 7 inside of the balloon 6. In the present embodiment, the radiofrequency transmission line 8 is formed from a coated copper wire, and the coiled electrode 7 is formed from a copper wire. The coiled electrode 7 is formed by peeling off the coating on the end of the radiofrequency transmission line 8.

Moreover, a metal wire 9 serving as a single superfine dissimilar metal wire formed from a metal dissimilar from the metal of the radiofrequency transmission line 8 which is sufficiently thick and capable of transmitting high currents, is brought into pin-point connection by means of welding, with the proximal end of the coiled electrode 7, that is, the connection part of the coiled electrode 7 and the radiofrequency transmission line 8. This metal wire 9 and the radiofrequency transmission line 8 constitute a thermocouple 10 which detects the temperature of the inside of the proximal end of the basal part of the coiled electrode 7. In the present embodiment, the metal wire 9 is formed from a coated constantan wire, and is exposed by peeling off the distal end only. Moreover, the metal wire 9 is made thinner than the radiofrequency transmission line 8.

In this manner, in the present embodiment, one of the dissimilar metal wires constituting the thermocouple 10 is used jointly as the radiofrequency transmission line 8. Consequently, compared to a conventional case where a thermocouple is independently arranged, one wire can be omitted, and thus the catheter can be miniaturized. Moreover, since the thermocouple formed by bringing the distal end of the metal wire 9 into pin-point connection with the proximal end of the coiled electrode 7, has a small heat capacity, then the temperature of the basal part of the coiled electrode 7 can be accurately and instantaneously detected.

A solution sending duct 11 is formed between the outer tube 2 and the inner tube 3, in communication with the inside of the balloon 6. Moreover, on the outside of the catheter shaft 1 is provided a vibration generator 21 which applies vibrational waves A to the balloon 6 via this solution sending duct 9. Swirl B are generated inside the balloon 6 by these vibrational waves A, so that the electrolytic solution inside the balloon 6 is agitated to keep the temperature inside the balloon 6 homogeneous.

Moreover, on the outside of the catheter shaft 1 is provided a radiofrequency generator 24 which supplies the coiled electrode 7 and a counter electrode plate 12 serving as the counter electrode thereof, with a radiofrequency. A low frequency cut filter 25 which cuts the low-frequency component of the radiofrequency output from the radiofrequency generator 24, is provided between the radiofrequency generator 24 and the radiofrequency transmission line 8 linking to the coiled electrode 7, and between the radiofrequency generator 24 and the counter electrode plate 12.

On the outside of the catheter shaft 1 is provided a thermometer 22 which indicates the temperature detected by the thermocouple 10. A high-frequency cut filter 23 which cuts the radiofrequency component input into the thermometer 22, is provided between the radiofrequency transmission line 8 and the metal wire 9 linking to the thermocouple 10, and the thermometer 22.

While the radiofrequency transmission line 8 is jointly used by these low frequency cut filter 25 and high-frequency cut filter 23, it becomes possible to eliminate noises due to radiofrequency currents, and to accurately measure the temperature by the thermocouple 10.

The radiofrequency generator 24 is designed to be capable of monitoring the radiofrequency output, the impedance, and reflection waves, while supplying the coiled electrode 7 and the counter electrode plate 12 with a radiofrequency of 1 to 5 MHz. By setting the number of cycles of radiofrequency to be supplied within a range of 1 to 5 MHz, efficient capacitive heating becomes possible around the balloon 6, and the total impedance that is the sum total of an internal balloon impedance being an impedance due to a liquid inside of the balloon 6, a membrane impedance being an impedance due to a membrane of the balloon 6, and a tissue impedance being an impedance due to biological tissue, can be accurately measured. If the number of cycles of radiofrequency to be supplied exceeds 5 MHz, the ratio of emission of radiofrequency power as electromagnetic waves is increased, and the impedance can not be accurately measured. If the number of cycles thereof is less than 1 MHz, the efficiency of the capacitive heating is remarkably decreased. Therefore, frequencies outside of the above range are unfavorable. As a result of experiments, it is found that the optimum value is 1.8 MHz.

With a conventional radiofrequency thermal balloon catheter system using a very high radiofrequency current (such as 13.5 MHz) for performing capacitive heating, with a lot of current leaks and it is not possible to measure the impedance. Whereas, in the present invention, the number of the cycle of radiofrequency current is greatly reduced from that of conventional systems, and the matching at the time of capacitive heating is accurately performed, thereby enabling capacitive heating by a balloon, even with the cycle of 1.8 MHz.

Moreover, the number of cycles of radiofrequency current is reduced to thereby suppress current leakage as much as possible, thereby enabling measurement of the total impedance that is the sum total of an internal balloon impedance, a membrane impedance, and a tissue impedance, and enabling detection of a pinhole in the balloon membrane and a thrombus on the balloon membrane.

Furthermore, the radiofrequency generator 24 comprises a control device (not shown) which automatically controls the radiofrequency output based on the temperature detected by the thermocouple 10, so that the temperature inside the balloon 6 can be kept at an electrode for delivery of radiofrequency energy. The radiofrequency generator 24 is designed to display by means of the control device, a pinhole alarm 26 as an alarm showing a pinhole occurrence in the membrane of the balloon 6 when the impedance is decreased by a fixed value with reference to a steady-state value, and to display a thrombus alarm 27 as an alarm showing a thrombus formation on the membrane of the balloon 6 when the impedance is increased by a fixed value with reference to a steady-state value. Alternatively, the radiofrequency generator 24 is designed to automatically stop supplying a radiofrequency current when the impedance is decreased or increased by a fixed value with reference to a steady-state value.

Next the operation of the radiofrequency thermal balloon catheter system of the present embodiment is described.

The lumen of the catheter shaft, that is, the inside of the solution sending duct 11 and the balloon 6, is filled with an electrolytic solution such as a physiological saline, to purge the air. Then, the outer tube 2 and the inner tube 3 are slid with each other so that the distance between the distal end 4 of the outer tube 2 and the distal end 5 of the inner tube 3 becomes a maximum, to thereby contract the balloon 6. Next, the elastic balloon 6 is placed in the treatment site. The distance between the distal end 4 of the outer tube 2 and the distal end 5 of the inner tube 3 is adjusted, and then the balloon 6 is expanded to abut against the treatment site.

Next, in order to make uniform the temperature distribution inside the balloon 6, vibration waves A are sent from the vibration generator 21 into the balloon 6. Then, a radiofrequency current is supplied from the radiofrequency generator 24 to start heating. The radiofrequency generator 24 automatically controls the output so that the temperature inside the balloon 6 can be kept at a target value. Consequently, even in a miniature catheter of which the temperature is relatively difficult to control due to a small heat capacity, heating can be efficiently performed while keeping the inside the balloon 6 at a target temperature. Then, the treatment site is cauterized at a predetermined temperature for a predetermined time.

Here, if for some reason the radiofrequency transmission line 8 and the metal wire 9 constituting the thermocouple 10 are disconnected, the electromotive force of the thermocouple 10 becomes zero, and the temperature can not be measured. Therefore, it is immediately found that the thermocouple 10 is faulty. In a conventional structure where the thermocouple is arranged independently from the radiofrequency energizing electrode, there has been a problem in that the detection temperature becomes inaccurate due to the detachment of thermocouple from the electrode for delivery of radiofrequency energy. However, this problem can be eliminated.

Moreover, if a pinhole occurs in the balloon 6, the impedance of the membrane of the balloon 6 is decreased, resulting in a decrease in the measurement value of the impedance. When the impedance is decreased by more than a fixed value with reference to a steady-state value, the radiofrequency generator 24 judges that a pinhole has occurred in the balloon 6, and displays the pinhole alarm 26, or automatically stops supplying a radiofrequency. Consequently, excessive cauterization due to the pinhole occurrence can be prevented.

Furthermore, when a thrombus is adhered on the balloon 6, the impedance is increased due to the thrombus, resulting in an increase in the measurement value of impedance. When the impedance is increased by more than a fixed value with reference to a steady-state value, the radiofrequency generator 24 judges that a thrombus is adhered on the balloon 6, and displays the thrombus alarm 27, or automatically stops supplying a radiofrequency. Consequently, thromboembolism due to the adhesion of thrombi can be prevented.

As described above, the radiofrequency thermal balloon catheter system of the present embodiment includes: a catheter shaft 1 comprising the outer tube 2 and the inner tube 3 which are slidable with each other; the balloon 6 provided between the distal end 4 of the outer tube 2 and the vicinity of the distal end 5 of the inner tube 3; the coiled electrode 7 serving as a unipolar electrode for delivery of radiofrequency energy provided inside of this balloon 6; the radiofrequency transmission line 8 connected to this coiled electrode 7; the thermocouple 10 which detects the temperature of the coiled electrode 7; the solution sending duct 11 formed between the outer tube 2 and the inner tube 3, in communication with the inside of the balloon 6; the vibration generator 21 which applies vibrational waves A to the balloon 6 via this solution sending duct 11; the thermometer 22 which indicates the temperature detected by the thermocouple 10; the high-frequency cut filter 23 which is provided between this thermometer 22 and the thermocouple 10, and cuts the radiofrequency component input into the thermometer 22; the radiofrequency generator 24 which supplies the radiofrequency transmission line 8 and the counter electrode plate 12 provided outside of the balloon 6, with a radiofrequency; and the low frequency cut filter 25 which is provided between this radiofrequency generator 24 and the radiofrequency transmission line 8, and cuts the low-frequency component of the radiofrequency output from the radiofrequency generator 24; and the thermocouple 10 comprises the radiofrequency transmission line 8 and the single metal wire 9 joined to the distal end of the radiofrequency transmission line 8.

Since the thermocouple 10 comprises the radiofrequency transmission line 8 and the single metal wire 9 joined to the distal end of the radiofrequency transmission line 8, then if the metal wire 9 is disconnected from the radiofrequency transmission line 8, the measurement becomes impossible and the breakdown can be immediately judged. Consequently, the conventional problem in that, if for some reason the thermocouple is detached from the radiofrequency energizing electrode, the temperature will be detected in a location different from the location to be measured, and thereby the detection temperature becomes inaccurate, can be solved.

Moreover, by the above structure, one of the dissimilar metal wires constituting the thermocouple 10 is used jointly as the radiofrequency transmission line 8. Consequently, compared to a conventional case where a thermocouple is independently arranged, one wire can be omitted, and thus the catheter can be miniaturized by omitting the space for arranging the thermocouple.

Moreover, the coiled electrode 7 is formed in a coil shape by extending the distal end of the radiofrequency transmission line 8, and the distal end of the metal wire 9 is brought into pin-point connection with the proximal end of the coiled electrode 7. Therefore, the formation thereof is easy, and the connection part of the distal end of the metal wire 9 is reliably located inside of the balloon 6, and thus the detection temperature becomes accurate. Moreover, since the thermocouple formed by bringing the distal end of the metal wire 9 into pin-point connection with the proximal end of the coiled electrode 7, has a small heat capacity, the temperature of the basal part of the coiled electrode 7 can be accurately and instantaneously detected.

Furthermore, the radiofrequency generator 24 is designed to be capable of monitoring the radiofrequency output, the total impedance, and reflection waves, while supplying the coiled electrode 7 and the counter electrode plate 12 with a radiofrequency of 1 to 5 MHz. Furthermore, the radiofrequency generator 24 is designed to automatically control the radiofrequency output, so that the temperature of the coiled electrode 7 can be kept at a target value. Therefore, even if the catheter is miniaturized, the inside of the balloon can be efficiently heated and the handling can be facilitated.

Moreover, the radiofrequency generator 24 is designed to display the pinhole alarm 26, or to automatically stop supplying a radiofrequency, when the total impedance is decreased by a fixed value with reference to a steady-state value. Therefore, the phenomenon where a pinhole occurs and the impedance is decreased, can be reliably detected, and excessive cauterization can be prevented.

Furthermore, the radiofrequency generator 24 is designed to display the thrombus alarm 27, or to automatically stop supplying a radiofrequency, when the total impedance is increased by a fixed value with reference to a steady-state value. Therefore, the phenomenon where a thrombus is adhered and the impedance is increased, can be reliably detected, and thromboembolism can be prevented.

The present invention is not limited to the above embodiment, and various modifications can be made without departing from the scope of the present invention.

Hereunder is a description of experimental examples in a laboratory bath. In FIG. 2, reference symbol 31 denotes a laboratory bath. A phantom 32 resembling the shape of the treatment side, is set in the laboratory bath 31. The counter electrode plate 12 is set on the inner wall of the laboratory bath 31.

### EXPERIMENTAL EXAMPLE 1

The laboratory bath 31 was filled with a physiological salt solution at 37° C. to perform the experiment. The balloon was brought into contact with the phantom 32 in the physiological salt solution, and energized with a radiofrequency from the radiofrequency generator 24. Then, after about 100 seconds, the impedance, the output, the temperature of the center of the balloon 6, and the reflection waves were all in a steady-state. After 300 seconds from the start of energization, when a pinhole occurred in the balloon 6, although the output, the temperature, and the reflection waves slowly changed, the impedance was sensitively reacted and rapidly decreased.

### EXPERIMENTAL EXAMPLE 2

The laboratory bath 32 was filled with a heparinized whole blood solution at 37° C. to perform the experiment. The balloon was brought into contact with the phantom 32 in the whole blood, and energized with a radiofrequency from the radiofrequency generator 24, at a set temperature of 100° C. The temperature of the center of the balloon 6 was gradually increased, and when this exceeded 80° C. after about 300 seconds from the delivery of radiofrequency energy, a thrombus was formed on the surface of the balloon 6. Accompanying the thrombus formation, although the output, the temperature, and the reflection waves were slowly changed, the impedance was sensitively reacted and rapidly increased.

What is claimed:

1. A radiofrequency thermal balloon catheter system, including: a catheter shaft composed of an outer tube and an inner tube, which are slidable with each other; a balloon provided between a distal end of said outer tube and a vicinity of a distal end of said inner tube; a unipolar electrode for delivery of radiofrequency energy provided inside of this balloon; a radiofrequency transmission line connected to this unipolar electrode; a thermocouple which detects a temperature of said unipolar electrode; a solution sending duct formed between said outer tube and said inner tube, in communication with an inside of said balloon; a vibration generator which applies vibrational waves to said balloon via this solution sending duct; a thermometer which indicates a temperature detected by said thermocouple; a high-frequency cut filter which is provided between this thermometer and said thermocouple, and cuts a high-frequency component input into said thermometer; a radiofrequency generator which supplies said radiofrequency transmission line and a counter electrode plate provided outside of the balloon, with a radiofrequency; and a low frequency cut filter which is provided between this radiofrequency generator and said radiofrequency transmission line, and cuts a low-frequency component of the radiofrequency output from said radiofrequency generator; and
said thermocouple: is composed of said radiofrequency transmission line and a single superfine dissimilar metal wire joined to a distal end of said radiofrequency transmission line, and
said radiofrequency generator: is designed to be capable of monitoring a radiofrequency output, a total impedance that is the sum total of an internal balloon impedance, a balloon membrane impedance, and a tissue impedance, and reflection waves, while supplying said unipolar electrode and said counter electrode plate with a radiofrequency of 1 to 5 MHz: and further is designed to automatically control the radiofrequency output, so that the temperature of said unipolar electrode can be kept at a target value.

2. The radiofrequency thermal balloon catheter system according to claim 1, wherein said unipolar electrode is a coiled electrode formed in a coil shape by extending the distal end of said radiofrequency transmission line, and the distal end of said dissimilar metal wire is brought into pin-point connection with the proximal end of said coiled electrode.

3. The radiofrequency thermal balloon catheter system according to claim 1, wherein said radiofrequency generator is designed to indicate an alarm showing a pinhole occurrence in the membrane of said balloon, or to automatically stop supplying a radiofrequency current, when said total impedance is decreased by more than a fixed value with reference to a steady-state value.

4. The radiofrequency thermal balloon catheter system according to claim 1, wherein said radiofrequency generator is designed to indicate an alarm showing thrombus formation on the membrane of said balloon, or to automatically stop supplying a radiofrequency current, when the total impedance is increased by more than a fixed value with reference to a steady-state value.
